(19) United States
(12) Patent Application Publication
(10) Pub. No.: US 2012/0115780 A1
(43) Pub. Date: May 10, 2012

(54) Porogen Containing Calcium Phosphate Cement Compositions

(76) Inventors: David C. Delaney, Capitola, CA (US); Duran N. Yetkinler, San Jose, CA (US); Sahil Jalota, Santa Clara, CA (US); Ali Sait Ismailoglu, Menlo Park, CA (US); Ravinder Singh, San Jose, CA (US)

(21) Appl. No.: 13/250,713

(22) Filed: Sep. 30, 2011

Related U.S. Application Data
(60) Provisional application No. 61/389,023, filed on Oct. 1, 2010.

(51) Int. Cl. A61K 38/18; A61P 19/00; C04B 16/00; C04B 38/00; C04B 16/02

(52) U.S. Cl. 514/8.1; 106/672; 106/674; 106/677; 514/9.1

(57) ABSTRACT

Porogen containing calcium phosphate cement compositions are provided. Aspects of the cement compositions include a dry calcium phosphate reactant component, a setting fluid component and a porogen component. The porogen component includes at least first and second porogens having different pore forming profiles. Aspects of the invention include combining the cement components to produce a settable composition. Aspects of the invention further include the settable compositions themselves as well as kits for preparing the same. Methods and compositions as described herein find use in a variety of applications, including hard tissue repair applications.

### CROSS-REFERENCE TO RELATED APPLICATIONS

Pursuant to 35 U.S.C. §119 (e), this application claims priority to the filing dates of: U.S. Provisional Patent Application Ser. No. 61/389,023 filed on October 2010; the disclosure of which application is herein incorporated by reference.

### INTRODUCTION

Calcium phosphate cements find use as structural materials in the orthopedic and dental fields. Such cements are typically prepared by combining a dry component(s) and a liquid to form a flowable paste-like material that is subsequently capable of setting into a solid calcium phosphate product. Materials that set into solid calcium phosphate mineral products are of particular interest as such products can closely resemble the mineral phase of natural bone and are susceptible to remodeling, making such products extremely attractive for use in orthopedics and related fields.

While a large number of different calcium phosphate cement formulations have been developed, there is a continued need for the development of yet more advanced formulations.

### SUMMARY

Porogen containing calcium phosphate cement compositions are provided. Aspects of the cement compositions include a dry calcium phosphate reactant component, a setting fluid component and a porogen component. The porogen component includes at least first and second porogens having different pore forming profiles. Aspects of the invention include combining the cement components to produce a settable composition. Aspects of the invention further include the settable compositions themselves as well as kits for preparing the same. Methods and compositions as described herein find use in a variety of applications, including hard tissue repair applications.

### BRIEF DESCRIPTION OF THE FIGURES

FIG. 1 provides a Field-Emission Scanning Electron Microscope (FESEM) image showing microstructure and pore development of example containing α-tricalcium phosphate, calcium sulfate dihydrate, sodium chloride, and calcium alginate

FIG. 2 provides a FESEM image showing microstructure and pore to development of example containing alpha tricalcium phosphate, calcium sulfate dihydrate, sodium chloride, β-TCP, and calcium alginate.

FIG. 3 provides a FESEM image showing microstructure and pore development of example containing alpha tricalcium phosphate, calcium sulfate dihydrate, sodium chloride, β-TCP, and calcium alginate.

FIG. 4 provides a FESEM image showing microstructure and pore development of example containing α-tricalcium phosphate, calcium sulfate dihydrate, sodium chloride, β-TCP, polyglycolide, polyethylene glycol, and calcium alginate.

FIG. 5 illustrates the elution results of lysozyme over 7 day period from a composition prepared in Example 1 of the Experimental Section, below.

FIG. 6 illustrates the elution results of lysozyme over 14 day period from a composition prepared in Example 2 of the Experimental Section, below.

FIG. 7 illustrates the elution results of lysozyme over 14 day period from a composition prepared in Example 3 of the Experimental Section, below. FIG. 8 illustrates the elution results of lysozyme over 7 day period from a composition prepared in Example 4 of the Experimental Section, below.

FIG. 9 illustrates the elution results of lysozyme over 14 day period from a composition prepared in Example 5 of the Experimental Section, below.

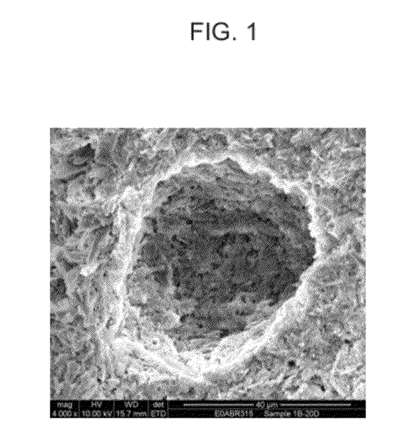

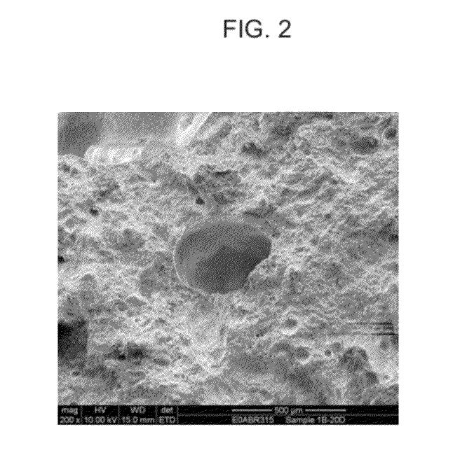

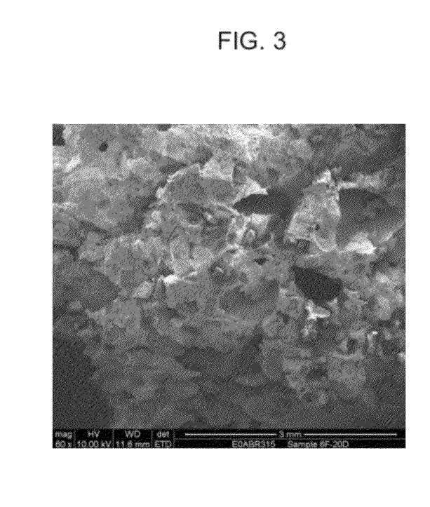

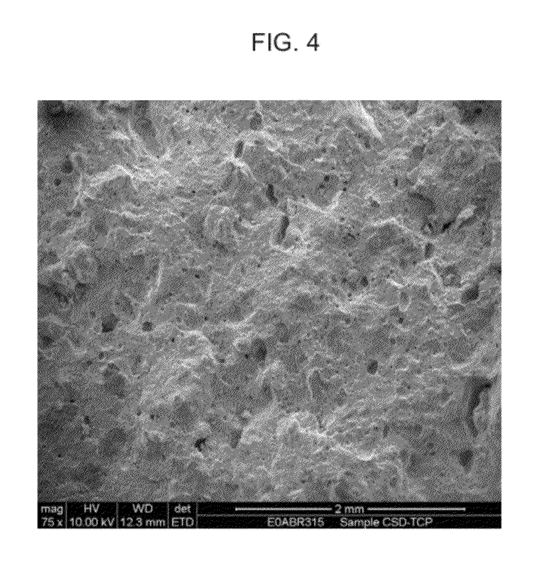

### DETAILED DESCRIPTION

Porogen containing calcium phosphate cement compositions are provided. Aspects of the cement compositions include a dry calcium phosphate reactant component, a setting fluid component and a porogen component. The porogen component includes at least first and second porogens having different pore forming profiles. Aspects of the invention include combining the cement components to produce a settable composition. Aspects of the invention further include the settable compositions themselves as well as kits for preparing the same. Methods and compositions as described herein find use in a variety of applications, including hard tissue repair applications.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided to may be different from the actual publication dates which may need to be independently confirmed.

### Porogen Containg Calcium Phosphate Cements and Settable Compositions Produced Therefrom

As summarized above, the porogen containing calcium phosphate cement compositions of the invention include the following components: a dry reactant calcium phosphate component comprising a calcium source and a phosphate source; a setting fluid component; and a porogen component. Each of these components is now described in greater detail.

### Dry Reactant Calcium Phosphate Component

The dry reactants include a calcium source and a phosphate source. The dry reactants may be particulate compositions, e.g., powders, where the particle size of the components of the particulate compositions may range from 1 to 1000 microns, such as from 1 to 200 microns and including from 1 to 40 microns.

As mentioned above, the dry reactants include a calcium source and a phosphate source. The calcium source and phosphate source may be present as a single compound or present as two or more compounds. As such, a single calcium phosphate present in the dry reactants may be the calcium source and the phosphate source. Alternatively, two or more compounds may be present in the dry reactants, where the compounds may be compounds that include calcium, phosphate or calcium and phosphate. Calcium phosphate sources of interest that may be present in the dry reactants include: MCPM (monocalcium phosphate monohydrate or Ca(H₂PO₄)₂.H₂O); DCPD (dicalcium phosphate dihydrate, brushite or CaHPO₄.2H₂O), ACP (amorphous calcium phosphate or Ca₃(PO₄)₂H₂O), DCP (or DCPA) (dicalcium phosphate, monetite or CaHPO₄), tricalcium phosphate (TCP), including both α- and β-(Ca₃(PO₄)₂, tetracalcium phosphate (Ca₄(PO₄)₂O, etc. Calcium sources of interest include, but are not limited to: calcium carbonate (CaCO₃), calcium oxide (CaO), calcium hydroxide (Ca(OH)₂) and the like. Phosphate sources of interest include, but are not limited to: Phosphoric acid (H₃PO₄), all soluble phosphates, and the like.

A variety of calcium phosphate cement compositions are known to those of skill in the art, and such cements may be readily modified into cements of the subject invention by including a porogen component, as described below. Cement compositions known to those of skill in the art and of interest include, but are not limited to, those described in U.S. Pat. Nos. 6,027,742; 6,005,162; 5,997,624; 5,976,234; 5,968,253; 5,962,028; 5,954,867; 5,900,254; 5,697,981; 5,695,729; 5,679,294; 5,580,623; 5,545,254; 5,525,148; 5,281,265; 4,990,163; 4,497,075; and 4,429,691; the disclosures of which are herein incorporated by reference.

The ratios or relative amounts of each of the disparate calcium and/or phosphate compounds in the dry reactant mixture is one that provides for the desired calcium phosphate product upon combination with the setting fluid and subsequent setting. In some embodiments, the overall ratio (i.e., of all of the disparate calcium and/or phosphate compounds in the dry reactants) of calcium to phosphate in the dry reactants ranges from 4:1 to 0.5:1, such as from 2:1 to 1:1 and including from 1.9:1 to 1.33:1.

In some instances, the dry reactants further include a monovalent cation dihydrogen phosphate salt. By monovalent cation dihydrogen phosphate salt is meant a salt of a dihydrogen phosphate anion and a monovalent cation, e.g., K+, Na+, etc., where the salt may or may not include one or more water molecules of hydration, e.g., may be anhydrous, a monohydrate, a dihydrate, etc. The monovalent cation dihydrogen phosphate salts present in the cements of these embodiments of the invention may be described by the following formula:

Y⁺H₂PO₄.(H₂O)ₙ

where:

Y⁺ is a monovalent cation, such as K+, Na+, etc.; and

n is an integer from 0 to 2.

In certain embodiments, the salt is a sodium dihydrogen phosphate salt, such as sodium biphosphate (i.e., sodium phosphate monobasic, NaH₂PO₄), or the monohydrate (NaH₂PO₄.H₂O) or dihydrate (NaH₂PO₄.2H₂O) thereof.

The amount of monovalent cation dihydrogen phosphate salt that is present in the dry reactants may vary, but is in some instances present in an amount sufficient to provide for a rapidly setting high strength attainment composition, as described in greater detail below. In certain embodiments, the salt is present in an amount that ranges from 0.10 to 10 wt. %, such as from 0.2 to 5.0 wt %, including from 0.5 to 2.0 wt. % of the total weight of the dry reactants. Further details regarding these salts and cements of interest that include the same are provided in United States Published Patent Application No. 20050260279, the disclosure of which is herein incorporated by reference.

In certain embodiments, the dry reactant portion or component of the cement includes a calcium and/or phosphate dry reactant that has a mean particle size (as determined using the Horiba LA-300 laser diffraction particle sizer (Version 3.30 software for Windows 95)(Irvine, Calif.)) of 8 μm or less and a narrow particle size distribution, e.g., as described in co-pending United States Published Patent Application 20070189951, the disclosure of which is herein incorporated by reference. As such, the dry reactant component of the cement, which may include one or more distinct dry reactants, includes a reactant that has a mean particle size of 8 μm or less and a narrow particle size distribution. The mean particle size of this reactant may vary, ranging in some embodiments from 1 to 7 μm, such as from 1 to 6 μm, including from 1 to 5 μm, where the mean particle size in certain embodiments may be 1, 2, 3 and 4 μm, where in certain embodiments the mean particle size is 3 μm.

This particular reactant of the subject cement compositions is further characterized in that it has a narrow particle size distribution. By narrow particle size distribution is meant that the standard deviation of the particles that make up the particular reactant population (as determined using the Horiba LA-300 laser diffraction particle sizer (Version 3.30 software for Windows 95)(Irvine, Calif.)) is 4.0 or less, and in certain representative embodiments is 3.0 or less, e.g., 2.5 or less, including 2.0 μm or less.

This particular reactant of the cement compositions of these embodiments may be further characterized in that the mode (as determined using the Horiba LA-300 laser diffraction particle sizer (Version 3.30 software for Windows 95)(Irvine, Calif.)) is 8.0 or less, such as 6.0 or less, e.g., 5 or less, including 3.0 μm or less.

In certain embodiments, the above described first reactant makes up the entire dry reactants of the composition, such that it makes up 100% of the dry component of the composition.

In certain embodiments, the dry reactants are further characterized by including a second reactant (a coarse particle reactant) that has a mean particle size that is 2 times or more larger than the mean particle size of the first reactant component, where the mean particle size of this second reactant may be 9 μm or larger, such as 10 μm or larger, including 20 μm or larger, e.g., 25 μm or larger, 30 μm or larger (as determined using the Horiba LA-300 laser diffraction particle sizer (Version 3.30 software for Windows 95)(Irvine, Calif.)) such as 50 μm or larger, 100 μm or larger, 150 μm or larger, 200 μm or larger, where the particle size of the tricalcium phosphate coarse particle component population (also referred to herein as a coarse particle size population) may range from 10 to 500 μm, such as from 25 to 250 μm. In certain instances, the particles of this component can range in size from 38 μm to 212 μm, such as from 38 μm to 106 μm or 106 μm to 212 μm. In some instances, this coarse particle component is manufactured using the protocol described in U.S. Published Patent Application No. 2010-0143480; the disclosure of which is herein incorporated by reference.

In certain embodiments, the amount of the first reactant component of the dry reactant composition is greater than the total amount of other reactant components that may be present, such as the second reactant component as described above. In certain of these embodiments, the mass ratio of the first reactant component to the total mass of the dry reactants may range from 1 to 10, e.g., from 9 to 6, such as from 9 to 7, including from 9.5 to 8.5.

In certain representative embodiments, the first reactant component is a calcium phosphate compound having a ratio of calcium to phosphate ranging from 1.0 to 2.0, including from 1.33 to 1.67, such as 1.5. In certain embodiments, the calcium phosphate compound is a tricalcium phosphate, such as α- and β-tricalcium phosphate, where in certain embodiments, the tricalcium phosphate is α-tricalcium phosphate.

In certain embodiments, the dry reactants may further include an amount of an emulsifying agent, as described in U.S. application Ser. No. 11/134,051 (published as US 2005-0260279); the disclosure of which is herein incorporated by reference in its entirety. Emulsifying agents of interest include, but are not limited to: polyoxyethylene or polyoxypropylene polymers or copolymers thereof, such as polyethylene glycol and polypropylene glycol; nonionic cellulose ethers such as methylcellulose, ethylcellulose, hydroxymethylcellulose, hydroxyethylcellulose, carboxymethylcellulose, carboxyethylcellulose and hydroxypropylcellulose; additional celluloses, such as carboxymethylcellulose sodium, carboxymethylcellulose calcium, carboxymethylstarch; polysaccharides produced by microbial fermentation, such as yeast glucans, xanthan gum, β-1,3-glucans (which may be straight-chained or branched; e.g. curdlan, paramylum, pachyman, scleroglucan, laminaran); other natural polymers, e.g., gum arabic, guar gum, carrageenin, gum tragacanth, pectin, starch, gelatin, casein, dextrin, cellulose; polyacrylamide; polyvinyl alcohol; starch; starch phosphate; sodium alginate and propylene glycol alginate; gelatin; amino-containing acrylic acid copolymers and quaternization products derived therefrom; and the like.

In certain embodiments, the emulsifying agent is a cellulose ether, particularly a nonionic cellulose ether, such as carboxymethylcellulose. Carboxymethylcellulose is available from a variety of commercial sources, including but limited to, Sigma, Hercules, Fluka and Noviant. In certain embodiments, the average molecular weight of the cellulose ether is 1000 daltons or higher, such as 5000 daltons or higher, where the average molecular weight may be as high as 10,000 daltons or higher, e.g., 50,000 daltons or higher, 100,000 daltons or higher, and ranges in certain embodiments from 5,000 to 100,000 daltons, such as from 10,000 to 50,000 daltons.

The proportion of the emulsifying agent in the dry reactant in certain embodiments ranges from 0.01 to 10% (w/w), such as from 0.05 to 2.0% (w/w).

### Setting Fluid Component

Another component of the cements is a setting fluid component. Setting fluids of interest include a variety of physiologically compatible fluids, including, but not limited to: water (including purified forms thereof, deionized forms thereof, etc.), aqueous alkanol solutions, e.g. glycerol, where the alkanol is present in minor amounts, e.g., 20 volume percent or less; pH buffered or non-buffered to solutions; solutions of an alkali metal hydroxide, acetate, phosphate or carbonate, particularly sodium, more particularly sodium phosphate or carbonate, e.g., at a concentration in the range of 0.01 to 2M, such as from 0.05 to 0.5M, and at a pH in the range of 6 to 11, such as from 7 to 9, including from 7 to 7.5; and the like.

In certain embodiments, a silicate setting fluid, i.e., a setting fluid that is a solution of a soluble silicate, is employed. By solution of a soluble silicate is meant an aqueous solution in which a silicate compound is dissolved and/or suspended. The silicate compound may be any compound that is physiologically compatible and is soluble in water. By soluble in water is meant a concentration of 1% or more, such as 2% or more and including 5% or more, where the concentration of the silicate employed may range from 0-0.1 to 20%, such as from 0.01-5 to 15% and including from 5 to 10%. Silicate setting fluids finding use with calcium phosphate cements are further described in U.S. Pat. No. 6,375,935; the disclosure of which is herein incorporated by reference.

In some embodiments, the setting fluid includes a cellulose component, such that the setting fluid is a cellulosic setting fluid. Of interest are water-soluble cellulose components, where specific cellulose components of interest include, but are not limited to: nonionic cellulose ethers, such as but not limited to: methylcellulose, ethylcellulose, hydroxymethylcellulose, hydroxyethylcellulose, carboxymethylcellulose, carboxyethylcellulose and hydroxypropylcellulose; additional celluloses, such as carboxymethylcellulose sodium, carboxymethylcellulose calcium, etc. In certain embodiments, the cellulose is carboxymethylcellulose. Carboxymethylcellulose is available from a variety of commercial sources, including but limited to, Sigma, Hercules, Fluka and Noviant. In certain embodiments, the average molecular weight of the cellulose is 1000 daltons or higher, such as 5000 daltons or higher, where the average molecular weight may be as high as 10,000 daltons or higher, e.g., 50,000 daltons or higher, 100,000 daltons or higher, and ranges in certain embodiments from 5,000 to 100,000 daltons, such as from 10,000 to 50,000 daltons. While the concentration of the cellulose in the setting fluid may vary, in some instances the concentration ranges from 0.5 to 5, such as 1 to 3 and including 2 to 3. In these instances, the setting fluid may be a fluid as described in U.S. patent application Ser. No. 12/771,999; the disclosure of which is herein incorporated by reference.

In some instances, the setting fluid is not a silicate setting fluid, i.e., the setting fluid does not include a silicate. As such, the setting fluid is not a silicate setting fluid as described in U.S. Pat. No. 6,375,935.

In certain embodiments, the setting fluid may further include an amount of phosphate ion, as described in U.S. Application Publication No. 20040250730; the disclosure of which is herein incorporated by reference in its entirety. For example, the concentration of phosphate ion in the setting fluid may vary, but may be 0.01 mol/L or greater, such 0.02 mol/L or greater and including 0.025 mol/L or greater, where the concentration may range from 0.01 to 0.5, such as from 0.01 to 0.25, including from 0.02 to 0.2 mol/L. The desired phosphate concentration may be provided using any convenient phosphate source, such as a non-calcium-containing salt of phosphoric acid that is sufficiently soluble, e.g., Na₃PO₄, Na₂HPO₄, or NaH₂PO₄. Salts of other cations such as K+, NH₄⁺, etc., may also be employed.

### Porogen Component

As summarized above, a third component of the calcium phosphate cements is a porogen component. Porogen components present in the calcium phosphate cements include at least a first porogen and a second porogen. The term “porogen” as used herein, refers to a chemical compound that is included in the settable composition produced upon combination of the cement components (e.g., as described in greater detail below). Upon implantation, the porogen is removed from the implanted composition, e.g., via diffusion, dissolution, and/or degradation, to leave a pore in the resultant at least partially implanted composition. A porogen may be viewed as an entity that reserves space in the settable composition while the composition is being prepared and implanted but once the composition is implanted the porogen is removed over time to result in porosity in the implanted composition, which may be at least partially set. In this way porogens provide latent pores in the settable composition.

As summarized above, the porogen component that is combined with the dry reactants and the setting fluid includes at least a first porogen and a second porogen. With respect to the first and second porogens, the first porogen has a pore forming profile that is shorter than the pore forming profile of the second porogen. A “pore forming profile” is a descriptor of the time at which half of the porogen in the composition is removed following implantation. In other words, if the time of implantation is T₀, then the pore forming profile is the time at which one half of the porogen has been removed from the implanted composition, where this time may be designated T_{1/2}. In some instances, the pore forming profile of the first porogen ranges 0.5 to 600 minutes, such as 1 to 300 minutes and including 1 to 160 minutes following implantation, e.g., 15 minutes to 120 minutes. The size of the pores produced by the first porogen may vary, ranging in some instances from 1 to 1000 μm, such as 1 to 500 μm and including 1 to 250 μm. In some instances, the first porogen may be viewed as a porogen that rapidly dissolves following implantation. First porogens of interest include both inorganic and organic porogens. Inorganic porogens of interest include, but are not limited to: inorganic salts, e.g., NaCl, MgCl₂, CaCl₂, NH₄Cl, NH₄PO₄, NH₄CO₃; soluble biocompatible salts; sugars (e.g., sugar alcohols), polysaccharides (e.g., dextran (poly(dextrose)), water soluble small molecules, natural or synthetic polymers, oligomers, or monomers that are water soluble or degrade quickly under physiological conditions, including but not limited to: polyethylene glycol, poly(vinylpyrollidone), pullulan, poly(glycolide), poly(lactide), poly(lactide-co-glycolide), other polyesters, and starches.

As reviewed above, the pore forming profile of the second porogen exceeds that of the first porogen. In some instances, the pore forming profile of the second porogen exceeds that of the first porogen by a factor of 50 or longer, such as 100 or longer, including 200 or longer, e.g., 500 or longer, including 1000 or longer. In some instances, the pore forming profile of the second porogen ranges from 60 minutes to 1 year, such as 60 minutes to 9 months, e.g., 60 minutes to 6 months, including 1 day to 6 months, e.g., 1 week to 6 months, as well as 1 month to 6 months following implantation. In some instances, the second porogen dissolves in a time-delayed manner upon implantation. The size of the pores produced by the second porogen may vary, ranging in some instances from 1 μm to 500 μm, such as 5 μm to 500 μm, e.g., 1 μm to 250 μm. Second porogens of interest may vary, wherein examples of such porogens include, but are not limited to: alginates (including cross-linked versions thereof), inorganic calcium compounds, e.g., calcium sulfate, β-TCP; chitosans, biodegradeable esters, e.g., polylactic acid (PLA), polycaprolactone (PCL), polyesteramide (PEA), etc.; polyoxyethylene or polyoxypropylene polymers or copolymers thereof, such as polyethylene glycol and polypropylene glycol; celluloses, e.g., nonionic cellulose ethers such as methylcellulose, ethylcellulose, hydroxymethylcellulose, hydroxyethylcellulose, carboxymethylcellulose, carboxyethylcellulose and hydroxypropylcellulose, and additional celluloses, such as carboxymethylcellulose sodium, carboxymethylcellulose calcium, carboxymethylstarch; polypropylene fumerate, bioactive glasses, e.g., bioglass; and the like.

The ratio of first to second porogen in the porogen component that is combined with the dry reactants and setting fluid to produce the settable composition may vary, ranging in some instances from 1:10 to 9:10, such as 1:8 to 7:8. In some instances, the amount of first porogen that is employed ranges from 1 to 20, such as 1 to 10% by weight. In some instances, the amount of second porogen that is employed ranges from 25 to 80, such as 50 to 75% by weight.

The porogen component, e.g., as described above, may be initially present as a component separate from the dry reactants and setting fluid components, or combined with one or both of these initially disparate components, such that it may be present in the dry reactants and/or setting fluid when the dry reactants and setting fluid are combined, as described below.

Where desired, the first and/or second porogens may be further processed into a desirable composition format, for example a three dimensional structural configuration. Examples of three dimensional structural configurations of interest include, but are not limited to: gel micro-beads, hollow spheres, fibers, foams, and the like. These structure may further be coated with various combinations of dissolution modulatory materials, e.g., organic or in organic layers, which adjust the disollution/resorption of the porogen following implantation.

### Additional Cement Components

One or both of the above liquid and dry reactant components may include an active agent that modulates the properties of the product into which the flowable composition prepared by the subject method sets. Such additional ingredients or agents include, but are not limited to: organic polymers, e.g., proteins, including bone associated proteins which impart a number of properties, such as enhancing resorption, angiogenesis, cell entry and proliferation, mineralization, bone formation, growth of osteoclasts and/or osteoblasts, and the like, where specific proteins of interest include, but are not limited to: osteonectin, bone sialoproteins (Bsp), α-2HS-glycoproteins, bone Gla-protein (Bgp), matrix Gla-protein, bone phosphoglycoprotein, bone phosphoprotein, bone proteoglycan, protolipids, bone morphogenic protein, cartilage induction factor, platelet derived growth factor, skeletal growth factor, and the like; particulate extenders; inorganic water soluble salts, e.g., NaCl, calcium sulfate; sugars, e.g., sucrose, fructose and glucose; pharmaceutically active agents, e.g., antibiotics; and the like. Of particular interest in certain embodiments are formulations that include the presence of one or more osteoinductive agents, including, but not limited to, those listed above. Additional active agents of interest include osteoclast induction agents, e.g., RANKL, as described in U.S. Pat. No. 7,252,833, the disclosure of which is herein incorporated by reference.

In some instances, an angiogenic factor is combined with the dry reactants and setting fluid, so that the flowable composition includes an amount of an angiogenic growth factor. As used herein, an “angiogenic growth factor polypeptide” refers to any protein, polypeptide, mutein or portion that is capable of inducing endothelial cell growth.

Angiogenic growth factors of interest include, but are not limited to: vascular endothelial cell growth factors (VEGF), acidic fibroblast growth factor (aFGF), basic fibroblast growth factor (bFGF), FGF2, epidermal growth factor, transforming growth factors α and β, platelet-derived endothelial growth factor, platelet-derived growth factor, tumor necrosis factor a, hepatocyte growth factor (scatter factor), erythropoietin, colony stimulating factor (CSF), macrophage-CSF (M-CSF), granulocyte/macrophage CSF (GM-CSF), angiopoietin 1 and 2, and nitric oxide synthase (NOS). The nucleic acid and amino acid sequences for these and other angiogenic growth factors are available in public databases such as GenBank and in the literature.

In some instance, the angiogenic growth factor is a VEGF, where VEGF proteins of interest include, but are not limited to: VEGF 1 (also referred to as VEGF A); VEGF 2 (also referred to as VEGF C); VEGF B; and VEGF D), PGF, etc. In addition to the above angiogenic growth factors, also of interest are their homologs and alleles and functionally equivalent fragments or variants thereof. For example, human VEGF 1 (VEGF A) exists in four principal isoforms, phVEGF₁₂₁; phVEGF₁₄₅; phVEGF₁₆₅; and phVEGF₁₈₉. Also of interest are the VEGF proteins and mutants thereof described in U.S. Pat. Nos. 5,851,989; 5,972,338; 057,428; 6,258,560; 6,348,351; 6,350,450; 6,368,853; 6,391,311; 6,395,707; 6,451,764; 6,455,496; 6,492,331; 6,551,822; 6,576,608; 6,586,397; 6,620,784; 6,750,044; 6,897,294; 6,927,024; 7,005,505; 7,060,278; 7,090,834; 7,208,472; 7,323,553; 7,427,596; 7,446,168; 7,494,977; 7,632,810; 7,651,703; 7,700,571; 7,709,455; 7,727,536; 7,785,588.

In some instances, the angiogenic factor (when present) may be complexed with an agent that modulates the release of the angiogenic factor from the settable composition following implantation, i.e., a release modulatory agent. By “complexed with” is meant that the angiogenic factor and the release modulatory agent are intimately associated with each other. The nature of the intimate association of the angiogenic factor and the release modulatory agent may vary, where examples of intimate associate include, but are not limited to: co-precipitation, encapsulation, dispersion, and the like, and may be achieved using a variety of different protocols, including but not limited to: co-precipitation, dip-coating, spray coating, solvent evaporation (lyophilization), etc.

The release modulatory agent may be any of a variety of different materials, so long as the materials are biocompatible and provide for the desired release modulatory activity. Release modulatory agent materials of interest include both inorganic and organic materials. Inorganic materials of interest include, but are not limited to: calcium phosphates, such as amorphous calcium phosphate crystalline hydroxyapatite, calcium sulphates, such as calcium sulphate dihydrate, calcium sulphate hemihydrate, etc. Organic materials of interest include, but are not limited to, organic polymers, e.g., alginates, chitosan, celluloses, PVA, PEG, gelatin, collagen, etc. Of interest are organic polymers that readily form gels and are cross-linkable at room or body temperature by common biocompatible methods.

In some instances, the release modulatory agent is a porogen of the porogen component, e.g., a first or second porogen of the porogen component. Examples of release modulatory agents that may also serve as porogens include, but are not limited to: calcium sulphate, algitate, chitosan, β-TCP and the like.

Where desired, the angiogenic factor/release modulatory agent complex may be further processed into a desirable composition format, for example a three dimensional structural configuration. Examples of three dimensional structural configurations of interest include, but are not limited to: gel micro-beads, fibers, foams, and the like.

In some instances, the angiogenic factor is employed in the absence of a cement that includes a porogen component. Accordingly, aspects of the invention further include calcium phosphate cements that include an angiogenic factor, e.g., as described above, but lack a porogen component.

In addition, in certain embodiments the compositions include demineralized bone matrix, which may be obtained typically in a lyophilized or gel form and is combined with the cement composition at some prior to implantation. A variety of demineralized bone matrixes are known to those of skill in the art and any convenient/suitable matrix composition may be employed.

Aspects of the invention include the presence of cyclodextrin in the composition prepared from the dry reactants and the setting fluid. Depending on the desired format, the cyclodextrin may be present in the dry reactants or in the setting fluid. By cyclodextrin is meant a cyclic oligosaccharide or mixture of cyclic oligosaccharides, composed of 5 or more α-D-glucopyranoside units that exhibit a 1->4 linkage. Cyclodextrins of interest include α-cyclodextrin, β-cyclodextrin and γ-cyclodextrin. The amount of cyclodextrin that is present in either the liquid or dry components may vary, depending on the amount that is desired in the flowable composition produced therefrom. In some instances, the amount of cyclodextrin that is desired in the flowable composition produced upon combination of the dry reactants and setting fluid ranges from 0.01 to 10% (w/w), such as 0.05 to 2.0% (w/w). In some instances where the cyclodextrin is present in the dry reactant component, the amount of cyclodextrin that is present in the dry reactant component ranges from 0.01 to 10% by weight, such as 0.05 to 2.0% by weight. Cyclodextrin components and details regarding the same are further described in U.S. patent application Ser. No. 12/568,531; the disclosure of which is herein incorporated by reference.

In certain embodiments, the cement may further include a contrast or imaging agent, where the contrast agent may be present in one or both of the liquid and dry components, or separate therefrom until combination of all of the components to produce the flowable composition. Contrast agents of interest include, but are not limited to: the water soluble contrast agents described in U.S. Pat. No. 7,306,786, the disclosure of which is herein incorporated by reference in its entirety; and the barium apatite contrast agents described in U.S. application Ser. No. 10/851,766 (Published as US20050257714), the disclosure of which is herein incorporated by reference in its entirety.

In certain embodiments, the subject cement compositions may be seeded with any of a variety of cells, as described in published U.S. Patent Publication No. 20020098245, the disclosure of which is herein incorporated by reference in its entirety.

### Methods of Combining Cement Components to Produce a Settable Composition

As reviewed above, in producing settable compositions of the invention that are suitable for implantation, suitable amounts of the dry reactants, the setting fluid and the porogen component are combined to produce the settable composition, where the settable composition sets into a solid product following implantation. The ratio of the dry reactants to setting fluid (i.e. the liquid to solids ratio) is selected to provide for an initial “flowable” composition that is also settable, where by “settable” is meant that the composition goes from a first non-solid (and also non-gaseous) state (i.e., flowable state) to a second, solid state after setting. In certain embodiments, the liquid to solids ratio is chosen to provide for a flowable composition that has a viscosity ranging from that of milk to that of modeling clay. As such, the liquids to solids ratio employed in the subject methods ranges in some instances from 0.2 to 1.0, such as from 0.3 to 0.6. Of interest in certain embodiments are methods that produce a paste composition, where the liquid to solids ratio employed in such methods may range from 0.25 to 0.5, such as from 0.3 to 0.45.

The amount of porogen component (i.e., the total amount of porogens, including first and second porogens) that is combined with the dry and liquid components, described above, is sufficiently great to provide for the desired porosity in the final product following implantation. The amount of porogen component that is present in the settable composition may vary, but in certain embodiments ranges from 10% to 75% by weight, such as from 10% to 50% by weight.

As mentioned above, the requisite amounts of dry reactants, setting fluid and porogen component (which may be separate from or present in one or both of the dry reactants and setting fluid) are combined under conditions sufficient to produce the settable composition. In some instances, the dry and liquid components are combined under mixing (i.e., agitation) conditions, such that a homogenous composition is produced from the dry and liquid components. Mixing may be accomplished using any convenient protocol, including manual mixing (e.g., as described in U.S. Pat. No. 6,005,162 and automated mixing (e.g., as described in WO 98/28068), the disclosures of which publications are herein incorporated by reference. Also of interest is vibratory mixing, e.g., as described in U.S. Pat. Nos. 7,261,717; 7,252,672 and 7,261,718, the disclosures of which are herein incorporated by reference.

The temperature of the environment in which combination or mixing of the dry and liquid components takes place is sufficient to provide for a product that has desired setting and strength characteristics, and may range from 0 to 50° C., such as from 15 to 30 ° C., including 15 to 25° C., e.g., 16 to 18.5° C. or 22.5 to 25° C. In certain instances, mixing occurs at a temperature that is: 15° C., 16° C., 17° C., 18° C., 19° C., 20° C., 21° C., 22° C., 23° C., 24° C. and 25° C., or a temperature in between any sequential two of these temperatures.

Mixing takes place for a period of time sufficient for a flowable composition to be produced, and may take place for a period of time ranging from 15 to 120 seconds, such as from 15 to 100 seconds and including from 15 to 60 seconds, e.g., 15 to 50 seconds, 15 to 30 seconds, etc.

### Settable Composition

The above-described protocols result in the production of a settable composition that is capable of setting into a calcium phosphate mineral product, as described in greater detail below, where the settable composition is characterized by including an amount of porogen component. The settable compositions produced by the above-described methods are compositions that set into a biologically compatible, and often resorbable and/or remodelable, product, where the product is characterized by including calcium phosphate molecules not present in the initial reactants, i.e., that are the product of a chemical reaction among the initial reactants.

Prior to setting, the settable compositions are flowable. The term “flowable” is meant to include paste-like compositions, as well as more liquid compositions. As such, the viscosity time of the subject flowable compositions, defined as time periods under which the mixed composition injects through a standard Luer-lok fitting after mixing, may range up to 10 minutes, such as up to 7 minutes, including up to 4 minutes. Of interest in certain embodiments are paste compositions that have an injectable viscosity that injects in a time period ranging up to 5 minutes, such as up to 4 minutes. Pastes that stay paste-like for longer period may be displaced by bleeding bone once implanted into the body, which create a blood interface between the cement and the bone prior to the cement hardening.

The compositions produced according to embodiments of the invention set into calcium phosphate mineral containing products. By “calcium phosphate mineral containing” product is meant a solid product that includes one or more, usually primarily one, calcium phosphate mineral. In certain embodiments, the calcium phosphate mineral is one that is generally poorly crystalline, so as to be resorbable and, often, remodelable, over time when implanted into a physiologically site. The calcium to phosphate ratio in the product may vary depending on particular reactants and amounts thereof employed to produce it, and in some instances ranges from 2:1 to 1.33:1, such as from 1.8:1 to 1.5:1 and including from 1:7:1 to 1.6:1. Of interest in certain embodiments are apatitic products, which apatitic products have a calcium to phosphate ratio ranging from 2.0:1 to 1.33:1, including both hydroxyapatite and calcium deficient analogs thereof, including carbonate substituted hydroxyapatite (i.e. dahllite), etc. The subject paste-like composition is, in certain embodiments, one that is capable of setting into a hydroxyapatitic product, such as a carbonated hydroxyapatite, i.e. dahllite, having a carbonate substitution of from 2 to 10%, usually from 2 to 8 by weight of the final product.

The period of time required for the compositions to harden or “set” may vary. Set time is determined using the Gilmore Needle Test (ASTM C266-89), modified with the cement submerged under 37° C. physiological saline. The set times of the subject cements may range from 30 seconds to 30 minutes, and will usually range from 2 to 15 minutes and more usually from 4 to 12 minutes. In certain embodiments, the settable composition sets in a clinically relevant period of time. By clinically relevant period of time is meant that the paste-like composition sets in less than 20 minutes, usually less than 15 minutes and often in less than 10 minutes, where the composition remains flowable for 1 minute or longer, usually 2 minutes or longer and, in many embodiments, for 5 minutes or longer following combination or mixture of the precursor liquid and dry cement components.

In some instances, the compositions rapidly set into a high strength product, as determined by the ASTM 0403/0403M-06 modified test described in U.S. patent application Ser. No. 12/771,999; the disclosure of which is herein incorporated by reference. In some instances, the compositions attain high strength rapidly, such that they may be viewed as rapid strength attainment compositions. As such, at 4 minutes the compositions of certain embodiments have a setting value of 150 Newtons or greater, e.g., 1000 Newtons or greater, such as 1200 Newtons or greater, where the setting value may be as high as 1300 or 1400 Newtons or greater, e.g., 1500 Newtons or greater, where in some embodiments the setting strength at 4 minutes ranges from 150 to 1500 Newtons. At 6 minutes the compositions may have a setting value of 1500 Newtons or greater, such as 1700 Newtons or greater, including 1800 Newtons or greater, e.g., 1900 Newtons or greater or 2000 Newtons or greater.

The compressive strength of the product into which the settable composition sets may vary significantly depending on the particular components employed to produce it. Of particular interest in many embodiments is a product that has a compressive strength sufficient for it to serve as at least a cancellous bone structural material. By cancellous bone structural material is meant a material that can be used as a cancellous bone substitute material as it is capable of withstanding the physiological compressive loads experienced by compressive bone under at least normal physiological conditions. As such, the subject flowable paste-like material is one that sets into a product having a compressive strength of 2 MPa or greater, e.g., 5 MPa or greater, including 10 MPa or greter, e.g., 15 MPa or greater, where in some embodiments the compressive strength is 20 MPa or greater, such as 40 MPa and greater, and including 50 MPa or greater, where in some instances the compressive strength ranges from 2 to 50 MPa, as measured by the assay described in Morgan, EF et al., 1997, Mechanical Properties of Carbonated Apatite Bone Mineral Substitute: Strength, Fracture and Fatigue Behavior. J. Materials Science: Materials in Medicine. V. 8, pp 559-570., where the compressive strength of the final apatitic product may be as high as 60 MPa or higher. Compressive strengths can be obtained that range as high 100 to 200 MPa.

The resultant product may have a high tensile strength. Tensile strength is determined using the protocol described in U.S. patent application Ser. No. 12/771,999 (the disclosure of which is herein incorporated by reference), and where the products may exhibit a 24-hour tensile strength of 0.5 MPa or greater, e.g., 1 MPa or greater, including 2.5 MPa or greater, e.g., 5 MPa or greater, such as 7 MPa or greater, e.g., 7.5 to 8 MPa, where in some instances the tensile strength ranges from 0.5 to 6.0 MPa.

In certain embodiments, the resultant product is stable in vivo for extended periods of time, by which is meant that it does not dissolve or degrade (exclusive of the remodeling activity of osteoclasts) under in vivo conditions, e.g., when implanted into a living being, for extended periods of time. In these embodiments, the resultant product may be stable for 4 months or longer, 6 months or longer, 1 year or longer, e.g., 2.5 years, 5 years, etc. In certain embodiments, the resultant product is stable in vitro when placed in an aqueous environment for extended periods of time, by which is meant that it does not dissolve or degrade in an aqueous environment, e.g., when immersed in water, for extended periods of time. In these embodiments, the resultant product may be stable for 4 months or longer, 6 months or longer, 1 year or longer, e.g., 2.5 years, 5 years, etc.

In certain embodiments of interest, the product that is produced is a composite product, which includes some unreacted particles, e.g., from the coarse particulate reactant, present in the final product. In certain of the embodiments where such a cement is implanted into an in vivo site, the unreacted particles may dissolve (e.g., via resorption) over time leaving a porous structure at the implant site, where the porous structure remains until it is remodeled. In certain embodiments, the remaining coarse particles in the composite may have a different radiopacity than the remainder of the product, e.g., where at least a portion of the coarse particles in the cement were dolomite.

In certain embodiments, the flowable paste-like settable composition is capable of setting in a fluid environment, such as an in vivo environment at a bone repair site. As such, the flowable paste composition can set in a wet environment, e.g., one that is filled with blood and other physiological fluids. Therefore, the site to which the flowable composition is administered during use need not be maintained in a dry state.

In those embodiments of the composition that including a porogen component, e.g., as described above, the implanted compositions have a porosity profile that is determined by the porogen component, e.g., by the first and second porogens present in the settable composition. The phrase “porosity profile” as used herein describes the nature of the porosity in the final product following setting, wherein in some instances the porosity profile may also refer to the time period over which the pores form, i.e., how long it takes for the pores to form following implantation (i.e., T₀).

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some instances, porosity (e), defined as the volume fraction pores, can be calculated from composite foam density, which can be measured gravimetrically.

In some instances, the porosity profile of a set composition includes a collection of micropores and macropores present in the composition following a predetermined amount of time following implantation of the material. Micropores are pores having a diameter ranging from 0.1 to 1 μm, such as 0.1 to 0.5 μm. Macropores are pores having a diameter ranging from 1 to 1000 μm, such as 1 to to 500 μm. As both micropores and macropores are present, the composition is both macroporous and microporous following a period of time after implantation. The ratio of micropores to macropores following a period of time after implantation may vary, ranges in some instances from 1:10 to 10:1. In some instances, the appearance of pores (micropores and/or macropores) in sufficient number to measurably impact (as measured by mercury porosimetry) the compressive and tensile strength of the implanted product does not occur for period of time following implantation of 24 hrs or longer.

In some instances, the settable compositions may be viewed as controlled pore forming calcium phosphate settable compositions. By “controlled pore forming” is meant that the calcium phosphate settable compositions assume a known porosity profile in a known amount of time following implantation and setting. In other words, the settable compositions assume a predetermined porosity profile in a known amount of time in situ following introduction to a body site, i.e., T₀.

In those embodiments where the settable composition includes a modifying factor, such as an angiogenic factor, the composition may be configured to release the factor from the composition for an extended period of time following implantation, i.e., T₀. In some instances, the factor is released for 2 days or longer, such as 5 days or longer, e.g., 1 week or longer, including 2 weeks or longer, such as 1 month or longer, following implantation. The amount of factor that is released over this period of time following implantation may vary, ranging from 10 mg/day or more, such as 15 mg/day or more, including 20 mg/day or more. In some instances, the release profile over this period of time is consistent, such that any variations in the amounts release over a given interval of the period (such as 12 hour period, 24 hour period, etc.) is 25% or less, such as 20% or less.

### Applications

Settable compositions produced from cements of the invention, e.g., as described above, find use in applications where it is desired to introduce a flowable material capable of setting up into a solid calcium phosphate product into a physiological site of interest, such as in dental, craniomaxillofacial and orthopedic applications. In orthopedic applications, the cement may be prepared, as described herein, and introduced or applied to a bone repair site, such as a bone site comprising cancellous and/or cortical bone. In some instances, the site of application is a cancellous bone void that results from reducing a fracture. In these instances, the methods may include reducing a bone fracture and then applying an amount of the flowable composition to the resultant void, where the amount may be sufficient to substantially if not completely fill the void.

Orthopedic applications in which the cements prepared by the subject system find use include, but are not limited to, the treatment of fractures and/or implant augmentation, in mammalian hosts, particularly humans. In such fracture treatment methodologies, the fracture is first reduced. Following fracture reduction, a flowable structural material prepared by the subject system is introduced into the cancellous tissue in the fracture region using the delivery device described above. Specific dental, craniomaxillofacial and orthopedic indications in which the subject invention finds use include, but are not limited to, those described in U.S. Pat. Nos. 6,149,655; 6,375,935; 6,719,993; 7,175,858; 7,252,833; 7,252,841; 7,252,672; 7,261,717; 7,306,786; 7,658,940; 7,658,940; and U.S. patent application Ser. Nos. 12/328,720; 12/568,531; and 12/771,999; the disclosures of which patents and patent applications are herein incorporated by reference in their entirety. In yet other embodiments, the subject compositions find use in drug delivery, where they are capable of acting as long lasting drug depots following administration to a physiological site. See e.g. U.S. Pat. Nos. 5,904,718 and 5,968,253; the disclosures of which are herein incorporated by reference in their entirety.

### Kits

Also provided are kits that include the subject cements, where the kits at least include a dry particulate component, a setting fluid, and a porogen component and/or an angiogenic factor, e.g., as described above. When both a dry component and setting fluid are present, the dry component and setting fluid may be present in separate containers in the kit, or some of the components may be combined into one container, such as a kit wherein the dry components are present in a first container and the liquid components are present in a second container, where the containers may or may not be present in a combined configuration, as described in U.S. Pat. No. 6,149,655, the disclosure of which is herein incorporated by reference. In addition to the cement compositions, the subject kits may further include a number of additional reagents, e.g., cells (as described above, where the composition is to be seeded), protein reagents (as described above), emulsifying agents, cyclodextrins, contrast agents, and the like.

In certain embodiments, the kits may further include mixing and/or delivery elements, e.g., mortar and pestle, spatula, etc., which elements find use in, e.g., the preparation and/or delivery of the cement composition.

In addition to above-mentioned components, the subject kits typically further include instructions for using the components of the kit to practice the subject methods. The instructional material may also be instructional material for using the cement compositions, e.g., it may provide surgical techniques and principals for a particular application in which the cement is to be employed. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Systems

Also provided are systems that find use in practicing the subject methods, as described above. The subject systems at least include dry and liquid components of a cement, as described above, and a mixing element. In certain embodiments, the systems may further include additional agents, e.g., contrast agents, active agents, etc., as described above.

The following examples are offered by way of illustration and not by way of limitation.

### EXPERIMENTAL

### Example 1

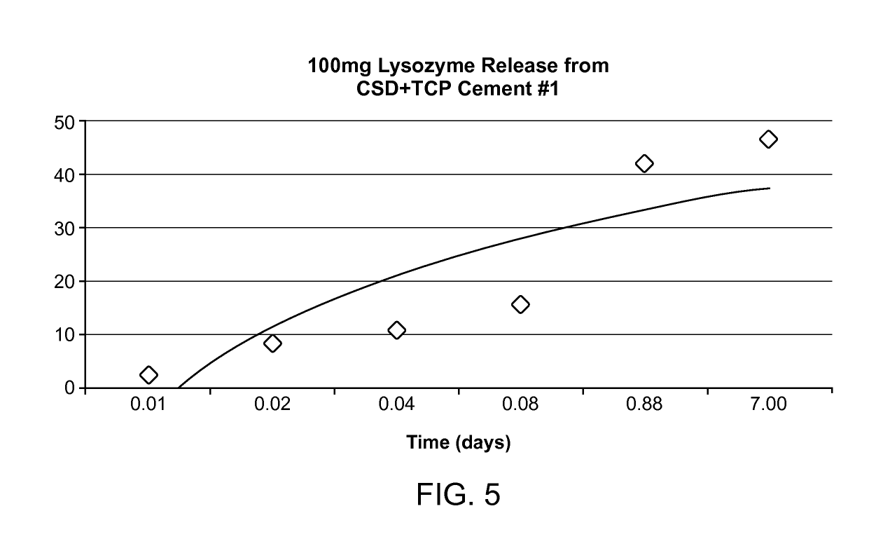

100 mg previously lyophilized lysozyme (purchased from Sigma) was added to 10 grams of sterile calcium sulfate hemihydrate (purchased from J. T. Baker) and dry blended for 30 minutes in a rotary blender. 3 mLs of sterile phosphate buffered saline was then spray added to the blending powders. The resulting paste was then dried in air, and the resultant dried particulate protein product was analyzed for calcium sulfate dihydrate and lysozyme concentrations using the following protocol XRD, FTIR, following EDTA dissolution. The resultant dried particulate protein product containing lysozyme and calcium sulfate was then added at 50% concentration by weight to α-tricalcium phosphate powder hydrated with phosphate buffered saline at a liquid to solids ratio of 0.45 to produce an injectable paste. The resultant paste was observed to harden within 20 minutes to a solid form hardened construct. Elution of lysozyme from this hardened construct was measured and found to be sustained past one week in vitro, as illustrated in FIG. 5. Dissolution of calcium sulfate is evidenced by pore generation over 15 days in vitro. Pores in the range of 1-500 μm are formed and seen when samples are imaged by SEM.

### Example 2

100 mg previously lyophilized lysozyme (purchased from Sigma) was added to 10 grams of sterile calcium sulfate hemihydrate (purchased from J. T. Baker) and dry blended for 30 minutes in a rotary blender. 3 mLs of sterile phosphate buffered saline was then spray added to the blending powders. The resulting paste was then dried in air, and analyzed by powder x ray diffraction followed by complete dissolution in EDTA for calcium sulfate dihydrate and lysozyme concentrations.

The resulting granular calcium sulfate material containing protein product from above was size classified and particles ranging from 100 to 1000 microns were then added to sterile aqueous solutions of the following:
a. 1% by weight sodium alginate (mol. weight 70K-200K Daltons) (purchased from FMC biopolymers)
b. 2% by weight sodium alginate mol weight (mol. weight 70K-200K Daltons) (purchased from FMC biopolymers)
c. 0.1% by weight sodium alginate mol weight (mol. weight 70K-200K Daltons) (purchased from FMC biopolymers)
d. 0.5% by weight sodium alginate mol weight (mol. weight 70K-200K Daltons) (purchased from FMC biopolymers)
e. 1% by weight chitosan gluconate mol weight (mol. weight 50K-250K Daltons) (purchased from FMC biopolymers)
f. 2% by weight chitosan gluconate mol weigh (mol. weight 50K-250K Daltons) (purchased from FMC biopolymers)

Soutions a-d were crosslinked upon contact with the calcium sulfate particles. Particles were then segregated and dried in air for 24 hrs.

Solutions e and f were added drop-wise into liquid nitrogen containing 2% glycerol phosphate, the resultant frozen beads collected, and lyophilized overnight.

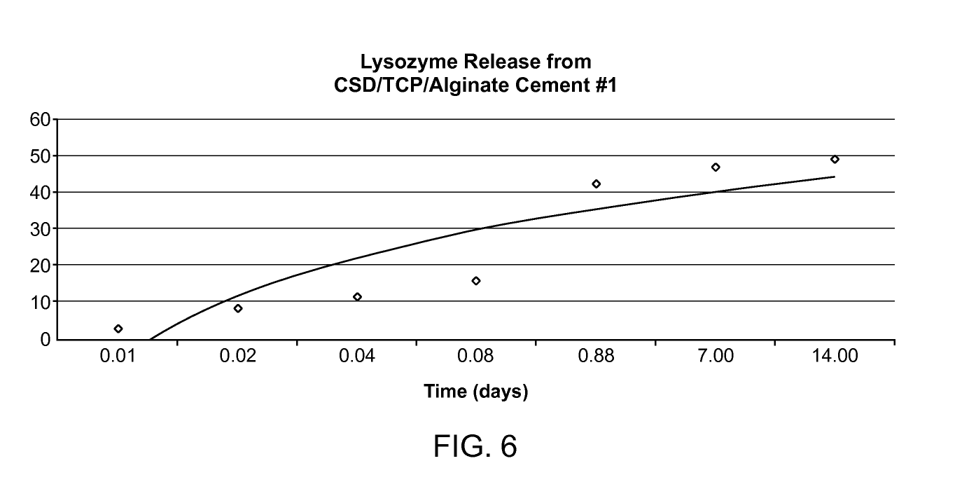

The dried particulate protein containing calcium sulfate complexed with either cross linked sodium alginate or lyophilized chitosan gluconate was then added at 50% concentration by weight to α-tricalcium phosphate powder (manufactured by Skeletal Kinetics, Cupertino Calif.) hydrated with phosphate buffered saline to produce an injectable paste that hardened within 20 minutes to a solid form hardened product. Elution of lysozyme from this hardened construct to was measured and showed sustained release past two weeks in vitro, as shown in FIG. 6. All samples showed elution of lysozyme past two weeks in vitro.

Dissolution of calcium sulfate is evidenced by pore generation over 15 days in vitro. Pores in the range of 1-500 μm are formed and seen when samples are imaged by SEM.

### Example 3

100 mg previously lyophilized lysozyme (purchased from Sigma) was added to 10 ml of 1% solution of sodium alginate (purchased from FMC biopolymers) and allowed to stir slowly for 15 minutes. Lysozyme-containing alginate solution was added drop-wise into a 100 mM calcium chloride solution (purchased from Sigma). Lysozyme-containing alginate beads formed instantaneously upon coming in contact with calcium chloride solution. These beads were collected from the solution, washed with deionized water and then lyophilized overnight. After lyophilization, the beads were analyzed for lysozyme concentration following lyophilization and elution of protein into PBS buffer.

100 mg previously lyophilized lysozyme (purchased from Sigma) was added to a 10 ml of 1% solution of sodium alginate (purchased from FMC biopolymers) and allowed to stir slowly for 15 minutes. To this solution, 1 gram of calcium phosphate powder (Fluidinova, calcium deficient hydroxyapatite nanoparticles) was added. This resultant solution was then added drop-wise into a 100 mM calcium chloride solution. Lysozyme-containing, calcium phosphate-incorporated alginate beads formed instantaneously upon coming in contact with calcium chloride solution. These beads were then collected from the solution, washed with deionized water and then lyophilized overnight. After lyophilization, the beads were analyzed for lysozyme concentration according to the following protocol: samples were dissolved in EDTA and dialysed against PBS buffer. Protein concentration was measured by BCA reagent (Fisher).

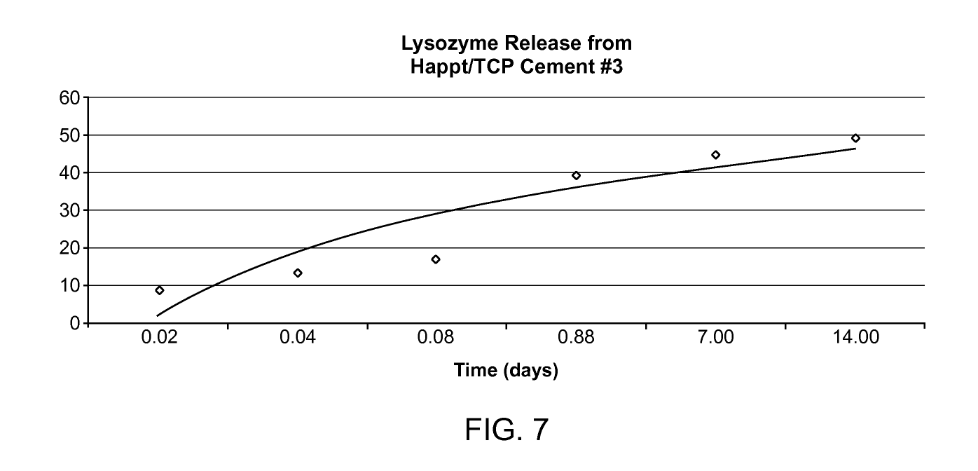

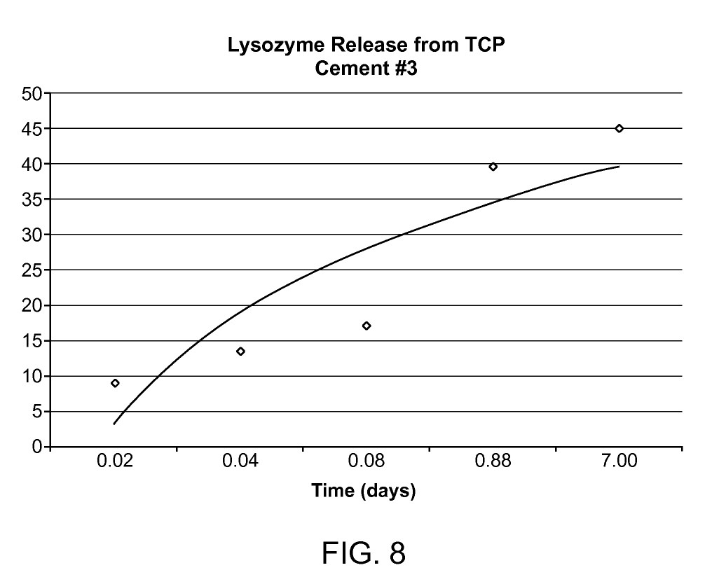

100 mg previously lyophilized lysozyme (purchased from Sigma) was added to 10 mLs of 0.1 M calcium acetate and allowed to stir for 10 minutes. To this solution was slowly added 5 mLs of 0.05M sodium phosphate with stirring while maintaining the pH of the solution at pH=7-8 by constant addition of sodium hydroxide. The resultant white precipitate was collected by filtration, lyophilyzed, and analyzed for lysozyme following dissolution in EDTA and dialysis against PBS buffer. This powder was stored under desiccation and later added at 5-25% concentration by weight to α-tricalcium phosphate powder (manufactured by Skeletal Kinetics) hydrated with phosphate buffered saline at a liquids to solids ratio of 0.42 to produce an injectable paste that hardened within 20 minutes to a solid form. Elution of lysozyme from this hardened construct was measured and showed sustained release past two weeks in vitro, as illustrated in FIG. 7.

Dissolution of calcium sulfate is evidenced by pore generation over 15 days in vitro. Pores in the range of 1-500 μm are formed and seen when samples are imaged by SEM.

### Example 4

100 mg previously lyophilized lysozyme (purchased from Sigma) was added to the powder component of a 5 cc Callos™/Scaffold™Bone Void filler kit (Skeletal Kinetics, Cupertino Calif.). The lysozyme-containing powder was mixed with the liquid from the kit according to the manufacturer's instructions to produce an injectable paste that hardened within 20 minutes to a solid form. Elution of lysozyme from this hardened construct was measured and showed sustained in vitro release past one week, as shown in FIG. 8.

Dissolution of calcium sulfate is evidenced by pore generation over 15 days in vitro. Pores in the range of 100-800 nm are formed and seen when samples are imaged by SEM.

### Example 5

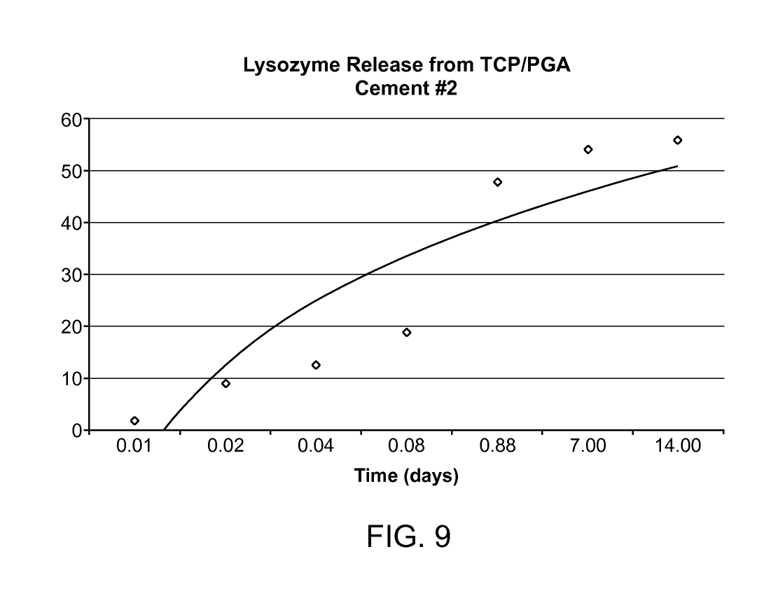

100 mg previously lyophilized lysozyme (purchased from Sigma) was added under high sheer mixing to a solution of 10% Poly lactic acid in Benzyl alcohol solvent (purchased from Sigma). This solution was then emulsified for 5 minutes in a 5% PVA solution (purchased from Sigma) to produce microspheres that were collected by filtration and solvent extraction. These microspheres were then stored under desiccation and later added at 5-25% concentration by weight to α-tricalcium phosphate powder (manufactured by Skeletal Kinetics) hydrated with phosphate buffered saline at a liquids to solids ratio of 0.4 to produce an injectable paste that hardened within 20 minutes to a solid form. Elution of lysozyme from this hardened construct was measured and showed sustained in vitro release past two weeks, as shown in FIG. 9.

Dissolution of calcium sulfate is evidenced by pore generation over 15 days in vitro. Pores in the range of 1-500 μm are formed and seen when samples are imaged by SEM.

### Example 6

A 5 cc cement kit is made by adding together the following components: 2 grams α-TCP (jet milled to a mean particle size of 3.2 μm, manufactured by Skeletal Kinetics), 1 gram NaCl (Sigma), 0.5 gram polyethylene glycol (m.w. 3400 Daltons, Sigma), 6 grams calcium sulfate dihydrate particle size range 20-500 μm (manufactured by Skeletal Kinetics) containing 25 mg recombinant human VEGF (manufactured by Roche/Genentech). The calcium sulfate dihydrate containing VEGF is made by the following procedure: 25 mg lyophilized VEGF is added to 3 cc sterile PBS and allowed to mix 15 minutes in a sterile field. This protein solution is added to 6 grams sterile calcium sulfate hemihydrate (J. T. Baker) to make a paste. The calcium sulfate hemihydrate containing VEGF is allowed to dry in air followed by light grinding to produce particles of calcium sulfate dihydrate in the size range described.

To the above powder component is added 3.0 grams of aqueous solution containing 2.0% sodium phosphate, pH=6.

The two components are mixed together to form an injectable paste with mortar pestle. The material is implanted in the medial aspect of the distal femoral metaphysis of 3 sheep. Immediately (within 10 minutes following implantation) pores are formed from the dissolution of PEG and NaCl into the surrounding fluid. The pore size ranges from 0.5 micron to 500 μm. Release of VEGF is apparent from histologies taken at 2 weeks and 1 month with the increased formation of blood vessels in and sometimes through the implanted material. Histological processing is by un-decalcified processing. Increasing pore formation throughout the material is observed at the 1 month and three month histologies as the calcium sulfate dihydrate is dissolved from the implant. The porosity is apparent from non decalcified histology sections and micro CT scanning results show pores ranging from 5-500 μm with some interconnected pores extending throughout the implant. Concurrent with pore formation and blood vessel invasion, new bone tissue is observed to fill the created pores as the implant becomes a composite structure of new bone architecture and remaining cement material.

All publications and patent applications mentioned in this specification are herein incorporated by reference to the same extent as if each individual publication or patent application was specifically and individually indicated to be incorporated by reference.

The invention now being fully described, it will be apparent to one of skill in the art that many changes and modifications can be made thereto without departing from the spirit and scope of the appended claims.

What is claimed is:

1. A method of producing a settable composition that sets into a calcium phosphate containing product, the method comprising:
combining:
(a) dry reactants comprising a calcium source and a phosphate source;
(b) a setting fluid; and
(c) a porogen component comprising:
(i) a first porogen; and
(ii) a second porogen;
in a ratio of (a), (b) and (c) sufficient to produce the settable composition;
wherein the first porogen has a pore forming profile that is shorter than the pore forming profile of the second porogen.

2. The method according to claim 1, wherein the first porogen has a pore forming profile ranging from 15 minutes to 120 minutes.

3. The method according to claim 2, wherein the first porogen is an inorganic salt.

4. The method according to claim 3, wherein the inorganic salt is selected from the group consisting of: NaCl, MgCl₂, CaCl₂, NH₄Cl, NH₄PO₄, NH₄CO₃ and combinations thereof.

5. The method according to claim 2, wherein the first porogen is an organic material.

6. The method according to claim 5, wherein the organic material is a polymer.

7. The method according to claim 1, wherein the second porogen dissolves in a time-delayed manner upon implantation.

8. The method according to claim 7, wherein the second porogen is an organic polymer.

9. The method according to claim 8, wherein the organic polymer is cross-linked.

10. The method according to claim 8, wherein the organic polymer is selected from the group consisting of: alginates, inorganic calcium compounds, celluloses, chitosans, biodegradeable esters, polyoxyethylene polymers, polyoxypropylene polymers, celluloses, polypropylen fumerate, bioactive glasses, and combinations thereof.

11. The method according to claim 1, wherein the method further comprises combining an angiogenic factor with the setting fluid, dry reactants and porogen is component.

12. The method according to claim 11, wherein the angiogenic factor is a VEGF or a FGF.

13. The method according to claim 12, wherein the VEGF is selected from the group consisting of: VEGF 1, VEGF 2, VEGF C, VEFG D, and combinations thereof.

14. The method according to claim 1, wherein said settable composition is a paste.

15. A kit comprising:
(a) dry reactants comprising a calcium source and a phosphate source;
(b) a setting fluid; and
(c) a porogen component comprising:
(i) a first porogen; and
(ii) a second porogen;
wherein the first porogen has a pore forming profile that is shorter than the pore forming profile of the second porogen.

16. The kit according to claim 15, wherein the first porogen rapidly dissolves upon implantation of the flowable material.

17. The kit according to claim 15, wherein the second porogen dissolves in a time-delayed manner upon implantation of the flowable material.

18. The kit according to claim 15, wherein the kit further comprises an angiogenic factor.

19. A settable composition that sets into a calcium phosphate containing product, wherein said composition is prepared by combining:
(a) dry reactants comprising a calcium source and a phosphate source;
(b) a setting fluid; and
(c) a porogen component comprising:
(i) a first porogen; and
(ii) a second porogen;
in a ratio of (a), (b) and (c) sufficient to produce the settable material;
wherein the first porogen has a pore forming profile that is shorter than the pore forming profile of the second porogen.

20. A method of repairing a hard tissue defect, the method comprising applying to the site of the defect a flowable composition according to claim 19.

21. A settable composition that sets into a calcium phosphate containing product, wherein said composition is prepared by combining:
(a) dry reactants comprising a calcium source and a phosphate source;
(b) a setting fluid; and
(c) an angiogenic factor;
in a ratio of (a), (b) and (c) sufficient to produce the settable material.

22. The settable composition according to claim 21, wherein the angiogenic factor is released from the settable composition for an extended period of time following implantation.
